(19) United States
(12) Patent Application Publication
(10) Pub. No.: US 2018/0011989 A1
(43) Pub. Date: Jan. 11, 2018

(54) ADJUSTING OVERALL DURATION OF PHYSICAL ACTIVITY

(71) Applicant: KONINKLIJKE PHILIPS N.V., EINDHOVEN (NL)

(72) Inventors: Gianluca Rospo, Eindhoven (NL); Francesco Sartor, Eindhoven (NL); Alberto Giovanni Bonomi, Eindhoven (NL)

(21) Appl. No.: 15/545,119

(22) PCT Filed: Jan. 22, 2016
(86) PCT No.: PCT/EP2016/051322

§ 371 Date: Jul. 20, 2017

(30) Foreign Application Priority Data
Jan. 23, 2015 (EP) 15152316.4

(51) Int. Cl. G06F 19/00; A61B 5/024; A61B 5/11; G06Q 10/06

(52) U.S. Cl. CPC G06F 19/3481; G06Q 10/0639; A61B 5/024; A61B 5/1112

(57) ABSTRACT

The invention provides a method, a system and a computer program product to adjust an overall duration of a physical activity of a biological object. The physical activity is evaluated based on a physical activity parameter. The invention includes receiving: a target information (S1) corresponding to the physical activity parameter; the overall duration (S2) being a sum of a plurality of distributed time intervals; and a physical activity parameter information of the biological object. The physical activity parameter information is received (S4) for each distributed time interval. Thereafter, the corresponding distributed time interval is modified (S5) based on the received physical activity parameter information and the target information. Subsequently, a new overall duration (S8) of the physical activity is determined based on the modified distributed time interval and the received overall duration.

### FIELD OF THE INVENTION

The invention relates to a method and system for adjusting an overall duration of a physical activity of a biological object, such as a person. The invention further relates to a computer program product for adjusting the overall duration of the physical activity of the person.

### BACKGROUND OF THE INVENTION

The importance of physical activity/exercising is well established. People like to maintain their health in today's fast paced life. One of the means to maintain health is to engage regularly in a physical activity. Various examples of the physical activity include but are not limited to running, jogging, body workouts in the gym, and the like. It is well known that performance of a user engaged in the physical activity can be measured based on various physical activity parameters associated with the person. Various examples of these parameters include but are not limited to a heart rate (HR), a pulse rate, oxygen consumption, power, and the like. Typically, these parameters are monitored for a pre-defined duration, for instance 30 minutes, corresponding to the physical activity. Further, there are also guidelines established by various health experts that also dictate a zone in which the physical activity parameter information, such as HR, has to be maintained. For instance, the user may be advised to maintain the heart rate in the zone of 90-110 beats-per-minute (bpm).

Solutions available currently in the state of the art do help the users to understand if the user was able to maintain the HR in the zone for the duration of the exercise. However, the working principles are not efficient. WO2008/003830A1 discloses a methodology wherein the exercise duration in a particular intensity, such as heart rate zone, can be varied based on a performance parameter, such as heart rate. The exercise duration is changed based on the performance parameter. Further, a pre-defined range is added or subtracted from the duration of the exercise. Thus, if the exercise is of 30 minutes, it can either be 24 or 36 (+/− 6 minutes). The disadvantage to such a methodology is that the person can be very close to his upper limit of the desired intensity, or very close to lower level of the intensity, however the pre-defined range is not added to the duration until and unless the person is either above/below a pre-defined threshold for the intensity. Thus, though the exercise duration is dynamic, the person may still land up to exercise over/under the desired duration.

### SUMMARY OF THE INVENTION

It is an object of the present invention to provide an improved method, a system for adjusting an overall duration of a physical activity of a person and a computer program product for implementing the improved method.

According to a first aspect of the invention this object is achieved by a method for adjusting an overall duration of a physical activity of a biological object, the overall duration being pre-defined, the physical activity being evaluated based on a physical activity parameter, the method comprising: receiving a target information corresponding to the physical activity parameter; receiving the overall duration, the overall duration being a sum of a plurality of distributed time intervals; receiving a physical activity parameter information of the biological object, the physical activity parameter information being received for each distributed time interval; modifying the corresponding distributed time interval based on the received physical activity parameter information and the target information, the corresponding distributed time interval being modified if the received physical activity parameter information deviates from the target information; and determining a new overall duration of the physical activity based on the modified distributed time interval and the received overall duration.

The method as described above provides numerous advantages over the existing methodologies. Firstly, given that the overall duration can be changed provides the advantage that the user is not over/under trained. The overall duration is modified during/while the person is exercising. Secondly, the methodology is accurate and precise because the modification of the overall duration is performed by modifying each distributed time interval. Various examples of physical activity parameter include but are not limited to a heart rate, a pulse rate, oxygen consumption, a breathing rate, speed, and power.

In a further embodiment of the invention, the time interval is modified in proportion to the difference between the target information and the received physical activity parameter information.

In a preferred embodiment of the invention, the time intervals are evenly distributed, such as one second. In other words, each second is evaluated before modifying the overall duration. Thus, the subsequent modified duration is more precise and therefore beneficial to the end user. Though, the example provided above mentions one second, the other evenly distributed time intervals can be 0.5 to 5 seconds.

The invention further includes, in an embodiment, receiving a zone corresponding to the physical activity parameter, a lower and an upper boundary of the zone are defined by a minimum physical activity parameter threshold and a maximum physical activity parameter threshold respectively. For instance, a heart rate zone: 80-110 beats-per-minute (bpm).

In another embodiment of the invention, the zone corresponding to the physical activity parameter can be determined. In these current embodiments in which either zone is determined or received, corresponding distributed time interval is modified if the received physical activity parameter information is in the zone. This is in particular advantageous to evaluate the performance of the user in a particular zone. If the person is exercising outside the recommended zone, i.e. either above the maximum threshold or below the minimum threshold, then the corresponding overall duration is not altered. Therefore, such a check helps the user to ensure that he exercises in the recommended zone.

Further, in a yet alternate embodiment, though the distributed time interval is modified, it is considered for modifying the overall duration only if the received physical activity parameter information is in the zone. Essentially, it provides the same advantages as elucidated in the previous paragraph.

Another embodiment also provides an option to the user, in order to increase user-friendliness, to select the physical activity parameter that he wants to be evaluated for the physical activity. For instance, the user may want the exercise to be evaluated based on HR or speed. The method furthermore provides, for instance by displaying, the new overall duration to the user. In an embodiment of the invention, the methodology as described above is computer implemented.

According to second aspect of the invention, a system is provided for adjusting an overall duration of a physical activity of a biological object, the overall duration being pre-defined, the physical activity being evaluated based on a physical activity parameter. The system comprises:
an interface for:
i. receiving a target information corresponding to the physical activity parameter;
ii. receiving the overall duration, the overall duration being a sum of a plurality of distributed time intervals;
at least one sensor for detecting a physical activity parameter information of the biological object, the physical activity parameter information being detected for each distributed time interval; and
a processing unit for:
i. modifying the corresponding distributed time interval based on the detected physical activity parameter information and the target information, the corresponding distributed time interval being modified if the detected physical activity parameter information deviates from the target information; and
ii. determining a new overall duration of the physical activity based on the modified distributed time interval and the received overall duration.

In a further embodiment of the invention, the processing unit modifies the corresponding distributed time interval in proportion to the difference between the target information and the received physical activity parameter information.

In an embodiment, the interface is further configured to receive a zone corresponding to the physical activity parameter. Further, a lower and an upper boundary of the zone are defined by a minimum physical activity parameter threshold and a maximum physical activity parameter threshold respectively.

In an embodiment, the processing unit is further configured to determine a zone corresponding to the physical activity parameter, wherein a lower and an upper boundary of the zone are determined based on the target information and a pre-determined mathematical range corresponding to the target information.

In an embodiment of the invention, the processing unit modifies the distributed time interval if the received physical activity parameter information is in the zone.

The system also enables, preferably by the interface, the user to select the physical activity parameter corresponding to the physical activity. Various examples of interface include but are not limited to a Graphical User Interface (GUI), a touch screen, push buttons, etc. Various examples of the physical activity parameter include but are not limited to a heart rate, a pulse rate, oxygen consumption, a breathing rate, speed, and power.

In an embodiment, the system further includes an information unit to provide the new overall duration to the biological object. In an embodiment of the invention, the information unit can be one of a display unit or an audio unit or a haptic unit or a combination thereof.

The system, as described above can be embodied in a wearable device, such as wearable on wrist, chest, etc. Thus, all the measurements and calculation of the modified/new overall duration will be performed in the wearable device.

In an alternative embodiment, the system (as described above) can be split into two or more modules. In an embodiment, the wearable device can only receive the target information and overall duration and simultaneously measure the physical activity parameter information of the user. Thereafter, the processing of the information and subsequent calculation of the distributed time interval for determining the new overall duration can be performed by a remote server. In this embodiment the remote server can be in constant connection with the wearable device through a network, preferably wireless network. Subsequently, the wearable device can provide the information/the new overall duration to the user through the inbuilt information unit.

In a second embodiment, the functionalities of the server can be replaced by a wireless communication device, such as a mobile device. Thus, the wireless communication device can receive the measurements from the wearable device to perform the calculation of the overall duration. Thereafter this information can be either displayed on the mobile device or on the wearable device.

In a third embodiment, the system (as described above) can be embodied in a wireless communication device independently. There are various straps/bands available in the market that permit the users to strap on the wireless communication device, for instance on an arm/wrist using an armband/wristband respectively. In yet another embodiment of the invention, the system can be embodied in an exercising unit, such as a treadmill.

According to the third aspect of the invention, a computer program product is provided. The computer program product having computer readable program code embodied therein, when executed by a computer, causing adjusting an overall duration of a physical activity of a biological object, the overall duration being pre-defined, the physical activity being evaluated based on a physical activity parameter, in which the adjustment comprising: receiving a target information corresponding to the physical activity parameter; receiving the overall duration, the overall duration being a sum of a plurality of distributed time intervals; receiving a physical activity parameter information of the biological object, the physical activity parameter information being received for each distributed time interval; modifying the corresponding distributed time interval based on the received physical activity parameter information and the target information, the corresponding distributed time interval being modified if the received physical activity parameter information deviates from the target information; and determining a new overall duration of the physical activity based on the modified distributed time interval and the received overall duration. Various examples of physical activity parameter include but are not limited to a heart rate, a pulse rate, oxygen consumption, a breathing rate, speed, and power.

Preferred embodiments of the invention are defined in the dependent claims. It shall be understood that the claimed methods, system, computer program product have similar and/or identical preferred embodiments as the claimed method and as defined in the dependent claims.

### BRIEF DESCRIPTION OF THE DRAWINGS

These and other aspects of the invention will be apparent from and elucidated with reference to the implementations and embodiments described hereinafter, and with reference to the accompanying drawings, which serve merely as non-limiting specific illustrations exemplifying the more general concept.

FIG. 1 shows a method for adjusting an overall duration of a physical activity of a biological object according to the invention;

FIG. 2 shows a table depicting the determination of adjusted overall duration according to an embodiment of the invention;

FIG. 3 shows a graph of the received HR information for the embodiment of the invention;

FIG. 4 shows a system for adjusting an overall duration of a physical activity of a biological object according to the invention;

FIG. 5 shows an implementation of the system as a wearable device according an embodiment of the invention;

FIG. 6 shows another implementation of the system comprising a wearable device and a wireless communication device; and

FIG. 7 shows yet another embodiment of the system as an exercising unit.

### DETAILED DESCRIPTION OF THE EMBODIMENTS

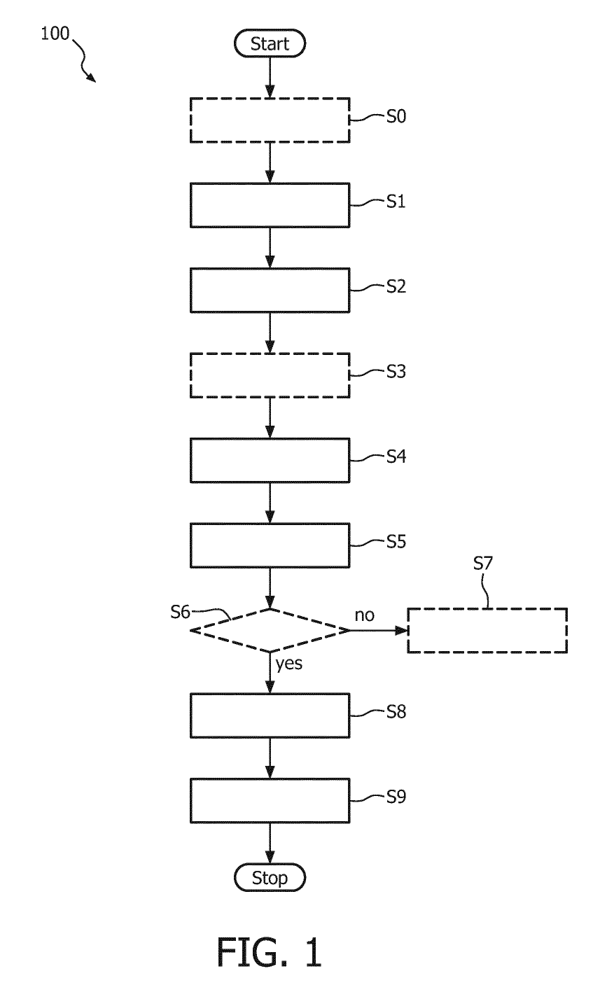

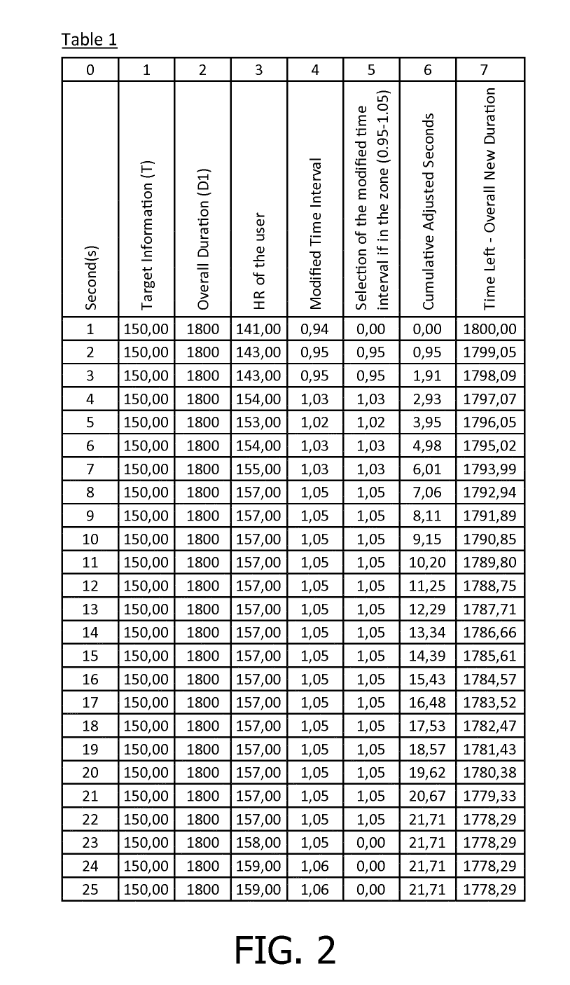

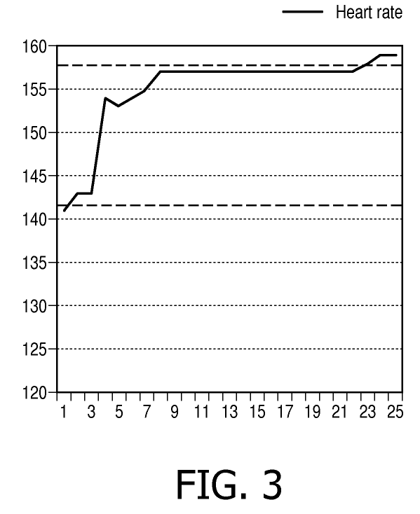

FIG. 1 shows a method 100 of adjusting an overall duration of a physical activity of a biological object according to the invention. The method has been explained with physical activity parameter as Heart Rate (HR). The method steps of FIG. 1 have been further explained in conjunction with FIG. 2 and FIG. 3.

The method 100 begins at S1 by receiving a target information corresponding to the HR. The target information is defined as the intended target that the user needs to maintain during the exercise, for instance 150 beats-per-minute (bpm). The target information is depicted in column 1 of Table 1 shown in FIG. 2. Optionally at S0 the method also provides selection of the physical activity parameter that the user wants to measure while engaging in a physical activity, such as running. Various other examples of physical activity parameter include but are not limited to a pulse rate, oxygen consumption, a breathing rate, speed, and power.

At S2, the overall duration of the physical activity is received. For instance, 30 minutes, i.e. 1800 seconds. The overall duration is depicted in column 2 of Table 1. The overall duration is the sum total of a plurality of distributed time intervals. In an embodiment of the invention, each time interval is evenly distributed. In the current example, one second is the evenly distributed interval. In other words, there are 1800 evenly distributed time intervals. In an embodiment of the invention, the user can provide the overall duration. In another embodiment of the invention, the overall duration corresponding to a physical activity parameter can be predefined. In yet another embodiment of the invention, each time interval need not be evenly distributed, for instance, the time intervals can be combination of 0.5, 1, and 3 seconds.

At S3, a zone corresponding to the HR is received. The zone, may also be referred to as a cardio zone, is defined by a lower and an upper boundary of the zone. The lower and upper boundaries correspond to a minimum HR threshold and a maximum HR threshold respectively. In the current example, the minimum HR threshold is 142.5 bpm and the maximum HR threshold is 157.5 bpm. In another embodiment of the invention, the zone can be determined based on the selected physical activity parameter. In the current example of HR parameter, the zone can be determined based on the target HR and a pre-determined mathematical range. For example, pre-determined mathematical range can be 5% and based on the received target information (150 bpm), then the upper and lower limit are determined as 157.5 bpm and 142.5 bpm respectively.

At S4, HR information of the user is received. In an embodiment of the invention, the HR information is received corresponding to each second (depicted in column 0 of Table 1), i.e. in real time, during the physical activity. In other words, for each second in the time duration, HR information is received. For instance, for first second the HR information is 141 bpm, for the second HR information is 143 bpm. HR information corresponding to each second is depicted in column 3 of Table 1. The received HR information (depicted as a solid line) is further depicted graphically in FIG. 3. The dotted lines in FIG. 3 represent the cardio-zone.

At S5, each second is modified based on the corresponding received HR information and the target information. The modification is performed by following formula:

Modified Time Interval=Received HR information/Target HR information.

Following the above examples, the modified time interval for the first second is 0.94 second. (141/150) and second one is 0.95 second (143/150). The modified intervals corresponding to each second are depicted in column 4 of Table 1. It should be evident that the above formula aims at modifying the time interval in proportion to the difference between the target information and the received physical activity parameter information.

In various embodiments of the invention, the corresponding second is modified if the received HR deviates from the target information. In other words, if the incoming HR information is 150 bpm then the corresponding second is not modified. Thus, the interval is counted/selected as one second only. In another embodiment of the invention, the time interval is modified even if the step S3 is not performed. In other words, the time interval can be modified for any HR information that deviates from the target information. In this embodiment, the user exercises irrespective of the zone information, thus making step 3 optional.

At S6, it is checked if the modified time interval is in the zone corresponding to the physical activity parameter (HR). In current embodiment, the zone is expressed in weighed maximum HR and weighed minimum HR. Following the above example, the upper boundary of the zone is 1.05 (157.5/150) and lower boundary of the zone is 0.95 (142.5/150). In another embodiment of the invention, the corresponding second is modified only if the received HR information is in the zone. Further, it may be apparent to a person skilled in the art that the zone can be also expressed directly in HR information, i.e. 142.2-157.5 bpm.

If the modified time interval, i.e. modified second, is not in the zone, then the modified time interval is discarded at S7. The modified time intervals that are discarded/ not considered for further calculations are depicted corresponding to second 1, second 23, second 24 and second 25 in column 5 of Table 1. The modified seconds are considered as zero (0). In another embodiment of the invention, the interval is modified only if the HR information is in the cardio zone and accordingly used for further calculations.

Further, if the modified second is in the zone, then it is considered for further determination, at S8, of new overall duration. The new overall duration can also be called as time remaining for the exercise to be completed. The considered modified seconds are depicted in column 5 of Table 1. Further, the new overall duration is 1799.05 (1800-0.95) is depicted in column 7 of Table 1. It can be observed that instead of reduction of ‘1’ second from the total duration, the duration is reduced by 0.95 second. Such a calculation is performed for each second and the new overall duration is calculated every second. It may be appreciated that in the Table 1 , the new overall duration is only calculated for first 25 seconds of 1800 seconds. Further, the first 25 seconds based on the methodology as described so far is calculated as 21.71 seconds. In other words, the duration 25 seconds is reduced by 3.29 seconds. S1milarly, by the end of the physical activity the user may not exercise for 1800 seconds but less or more based on the HR received corresponding to each second. In other words, when the HR is above the target HR a count-down timer will speed up its counting and vice versa when the HR is below a the target HR a count-down timer will slow down its counting. Thus, the current methodology calculates the new overall duration based on each heartbeat of the user.

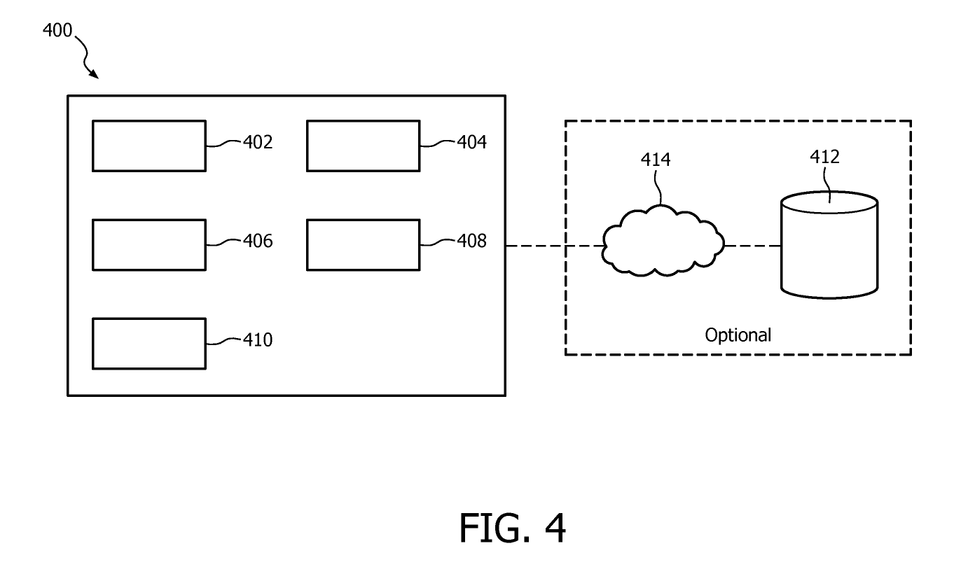

Thereafter, the new overall duration is provided to the user at S9. In an embodiment of the invention, the new overall duration can be provided to the user after each pre-defined interval. For example, after every 10 seconds, the new overall duration is provided to the user. In another embodiment of the invention, the new overall duration is provided real time to the user. Various ways in which duration is provided to the user is explained in detail in conjunction with FIG.4.

The method steps depicted within dotted lines in the flowchart are optional steps. For instance, S0, S3, S6, S7. It may be appreciated that these steps form alternate embodiments of the invention. Furthermore, steps such as S3 and S6 help to improve the overall accuracy of the new overall duration.

The method as described above, in an alternate embodiment, is embodied as a computer implemented method in which a computer or programmable processor is used which executes a computer readable program. Further the computer readable program is embodied in a computer program product, such as random access memory (RAM), read-only memory (ROM), hard disk drives, solid-state drives, USB flash drives, memory cards accessed via a memory card reader, floppy disks accessed via an associated floppy disk drive, optical discs accessed via an optical disc drive, magnetic tapes accessed via an appropriate tape drive, and/or other memory components, or a combination of any two or more of these memory components. In addition, the RAM may include, for example, static random access memory (SRAM), dynamic random access memory (DRAM), or magnetic random access memory (MRAM) and other such devices. The ROM may include, for example, a programmable read-only memory (PROM), an erasable programmable read-only memory (EPROM), an electrically erasable programmable read-only memory (EEPROM), or another like memory device. The computer program product can be an application (app) that can be installed on a computer, a wireless communication device, or a portable electronic device.

FIG. 4 shows an embodiment of a system 400 according to the invention for adjusting an overall duration of a physical activity of a biological object. The system 400 includes an interface 402, a sensor 404, a processing unit 406, a memory 408 and an information unit 410. In various embodiments of the invention, the system 300 is in close proximity of the user.

The interface 402 receives target information corresponding to a physical activity parameter. The interface 402 further receives an overall duration corresponding to the physical activity. In an embodiment of the invention, the interface 402 also receives zone corresponding to the physical activity parameter. The target information, the overall duration, the zone have been explained in detail in conjunction with FIG. 1. The interface 402 in an embodiment is a user interface (UI) to receive the target information and the overall duration directly from the user. Few examples of UI include but are not limited to a touch screen, push buttons, and a Graphical User Interface. In another embodiment of the invention, the interface 402 receives the target information and the overall duration directly from another device, such as a server, an application (app) installed on a wireless communication device, a browser running on a client device such a computer of the user/ health practitioner. In yet another embodiment of the invention, the target information and the overall duration corresponding to a physical activity parameter can be predefined.

The sensor 404 detects/receives physical activity parameter information of the user. In an embodiment of the invention, the sensor 404 is a heart rate sensor for detecting heart rate values/information of the user. An example of the heart rate sensor is a photoplethysmograph (PPG) sensor. Various other sensors 404 can be used based on the type of physical activity parameter that needs to be measured based on the user preferences. Few examples include but are not limited to a pulse rate sensor, a breathing sensor, O2 sensor, speed sensor, power/intensity sensor. In an embodiment of the invention, more than one sensor 404, such as mentioned above in any combination, can be used. The user can select via an interface 402 the type of physical activity parameter and accordingly the corresponding sensor 404 is used for receiving the physical activity information.

The processing unit 406 processes the received physical parameter information, for instance HR, along with the target information and modifies a distributed time interval corresponding to the received physical parameter information. Modification of the distributed time interval has been explained in detail above in steps S0-S5 of FIG. 1. Thereafter, the processing unit 406 checks if the modified time interval is in the zone. Subsequently, based on the check, the processing unit 406 calculates/determines the new overall duration based on the modified time interval and the overall duration. In an alternate embodiment of the invention, the processing unit 406 modifies the distributed time interval if the received physical parameter information is in the zone. Furthermore, the processing unit 406 executes steps S6-S8 as explained above to determine/calculate the new overall duration. It may be apparent to any person skilled in the art that the processing unit 406 may include a clock (not depicted in the figures) in order to countdown the remaining time (the new overall duration). In another embodiment of the invention, the clock may be a separate module in the system 400.

In another embodiment of the invention, the processing unit 406 determines zone corresponding to physical parameter chosen by the user. Such a determination has been explained above in conjunction with FIG. 1. Further, the system 400 includes the memory 408 to store the target information, the overall duration, the zone and such pre-determined ranges corresponding to different types of physical activity parameter. Further, the memory 408 may store instructions (S1-S9) that are executable by the processing unit 406 for calculation of the new overall duration. In an alternate embodiment of system 400, memory 412 (whole or in part) can also be outside the system 400 such as in cloud server architecture (depicted with dotted lines) and can be accessed over a network 414.

The term memory 408/412, as used herein, may include both volatile and/or nonvolatile memory and data storage components. Volatile components are those that do not retain data values upon loss of power. Nonvolatile components are those that retain data upon a loss of power. Thus, the memory may include, for example, random access memory (RAM), read-only memory (ROM), hard disk drives, solid-state drives, and/or other memory components, or a combination of any two or more of these memory components. In addition, the RAM may include, for example, static random access memory (SRAM), dynamic random access memory (DRAM), or magnetic random access memory (MRAM) and other such devices. The ROM may include, for example, a programmable read-only memory (PROM), an erasable programmable read-only memory (EPROM), and an electrically erasable programmable read-only memory (EEPROM), another like memory device. The storage unit 204 is a computer readable medium.

The term processing unit, as used herein, may be any type of controller or processor, and may be embodied as one or more controllers or processors adapted to perform the functionality discussed herein. Additionally, as the term processor is used herein, a processor may include use of a single integrated circuit (IC), or may include use of a plurality of integrated circuits or other components connected, arranged or grouped together, such as controllers, microprocessors, digital signal processors, parallel processors, multiple core processors, custom ICs, application specific integrated circuits, field programmable gate arrays, adaptive computing ICs, associated memory, such as and without limitation, RAM, DRAM and ROM, and other ICs and components.

The calculated new overall duration is provided to the user by the information unit 410. In an embodiment of the invention, the information unit 410 is one of a display unit, an audio unit, a haptic unit or a combination thereof The information unit 410, for instance by means of the display, can also display the remaining time that the user needs to exercise. Additionally, the current time spent in the cardio zone can also be simultaneously displayed. The information unit 410 can also provide analytical information, preferably by means of graphs, tables, etc., regarding various exercise sessions of the user.

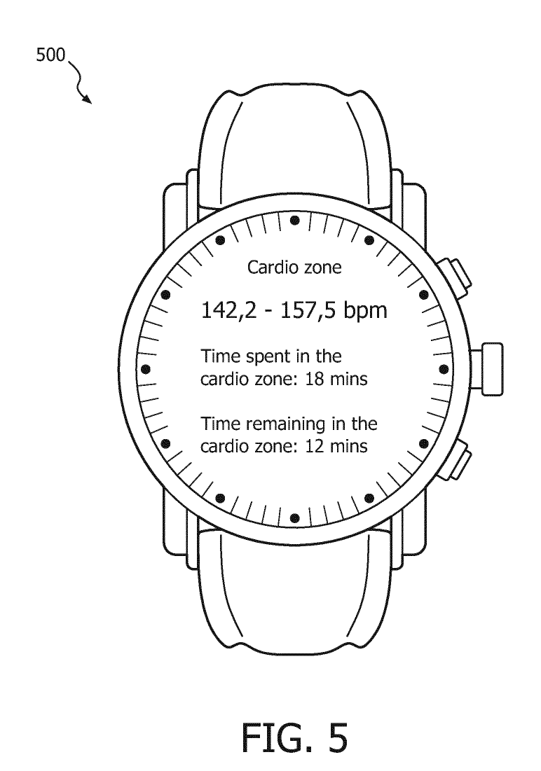

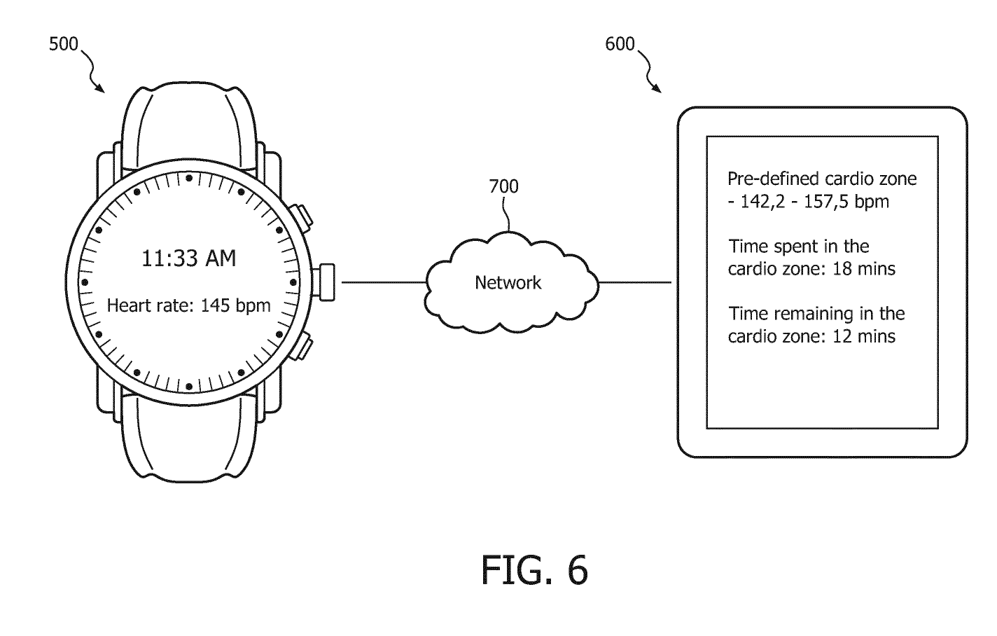

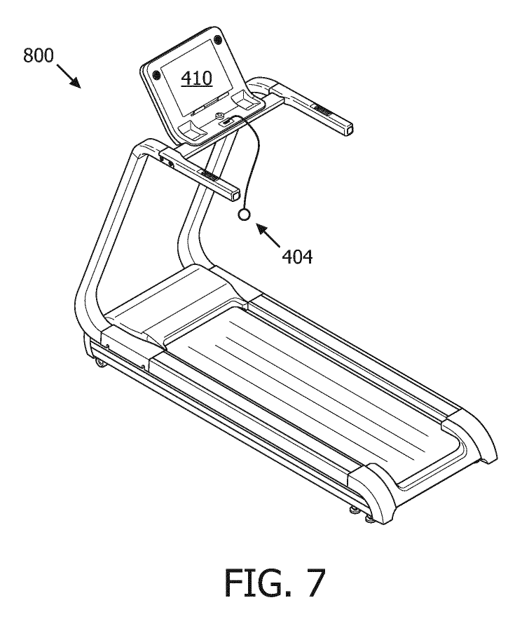

System 400 as described above can be embodied in as a wearable device, such as on a wrist, on the chest, etc. FIG. 5 shows an example of a wearable device 500 in the form of wrist based device. Such a wearable device 500 may interact with the user directly to receive the zone and to provide the new overall duration. However, the wearable device 500 may also interact simultaneously via an app installed on a wireless communication device 600 over a network 700 as depicted in FIG. 6. Thus, the app receives the zone and thereafter transmits the zone (information) to the wearable device 500. Subsequently, the wearable device 500 transmits the calculated duration to the app for further indication, such as in form display in an app, audio display, haptic feedback, and the like to the user. In yet another embodiment of the invention, the wearable device 500 may include only the sensor 404 and corresponding information can be transmitted over the network 700 to the app on the wireless communication device 600 for further calculation. In yet another embodiment of the invention, system 400 can be embodied into an exercising unit, such as a treadmill 800 (FIG. 7). Further, treadmill 800 includes at least one sensor 404 (wired/ wireless) that enables receiving the physical activity parameter information, for instance speed, of the user. Further, such a sensor 404 can be also to measure heart rate of the user. The treadmill 800 further includes the information unit 410 to provide the new overall duration to the user. It may be apparent to a person skilled in the art the treadmill 800 will further include other system components, which are not depicted in the FIG. 7, of the system 400. Further, treadmill 800 can also interact with the wireless communication device 600. It may be apparent to a person skilled in the art that various combinations as described above are possible without deviating from the scope of the invention.

Various examples of the wireless communication device 600 include but are not limited to a mobile device, a cellular telephone, a smart phone, a music player, a web pad, a tablet computer system, or other devices with like capability.

Various examples of the network 700/414 include but are not limited to the Internet, intranets, extranets, wired networks, wireless networks, wide area networks (WANs), local area networks (LANs), or other suitable networks, etc., or any combination of two or more such networks.

It will be clear to a person skilled in the art that the scope of the present invention is not limited to the examples discussed in the foregoing, but that several amendments and modifications thereof are possible without deviating from the scope of the present invention as defined in the attached claims. While the present invention has been illustrated and described in detail in the figures and the description, such illustration and description are to be considered illustrative or exemplary only, and not restrictive. The present invention is not limited to the disclosed embodiments.

Variations to the disclosed embodiments can be understood and effected by a person skilled in the art in practicing the claimed invention, from a study of the figures, the description and the attached claims. In the claims, the word “comprising” does not exclude other steps or elements, and the indefinite article “a” or “an” does not exclude a plurality. The mere fact that certain measures are recited in mutually different dependent claims does not indicate that a combination of these measures cannot be used to advantage. Any reference signs in the claims should not be construed as limiting the scope of the present invention.

1. A method for adjusting an overall duration of a physical activity of a biological object, the overall duration being pre-defined, the physical activity being evaluated based on a physical activity parameter, the method comprising:
receiving a target information, corresponding to the physical activity parameter;
receiving the overall duration, the overall duration being a sum of a plurality of distributed time intervals;
receiving a physical activity parameter information of the biological object, the physical activity parameter information being received for each distributed time interval;
modifying the corresponding distributed time interval based on the received physical activity parameter information and the target information, the corresponding distributed time interval being modified if the received physical activity parameter information deviates from the target information; and
determining a new overall duration of the physical activity based on the modified distributed time interval and the received overall duration.

2. The method according to claim 1, wherein the distributed time interval is modified in proportion to the difference between the target information and the received physical activity parameter information.

3. The method according to claim 1 further comprising receiving a zone corresponding to the physical activity parameter, a lower and an upper boundary of the zone are defined by a minimum physical activity parameter threshold and a maximum physical activity parameter threshold respectively, wherein the corresponding distributed time interval is modified if the received physical activity parameter information is in the zone.

4. The method according to claim 1 further comprising determining a zone corresponding to the physical activity parameter, wherein a lower and an upper boundary of the zone are determined based on the target information and a pre-determined mathematical range corresponding to the target information, wherein the corresponding distributed time interval is modified if the received physical activity parameter information is in the zone.

5. The method according to claim 1 further comprising receiving a zone corresponding to the physical activity parameter, a lower and an upper boundary of the zone are defined by a minimum physical activity parameter threshold and a maximum physical activity parameter threshold respectively, wherein the modified distributed time interval is considered for determining the new overall duration if the received physical activity parameter information is in the zone.

6. The method according to claim 1 further comprising determining a zone corresponding to the physical activity parameter, wherein a lower and an upper boundary of the zone are determined based on the target information and a pre-determined mathematical range corresponding to the target information, wherein the modified distributed time interval is considered for determining the new overall duration if the received physical activity parameter information is in the zone.

7. The method according to any of the preceding claims, wherein the distributed time intervals are evenly distributed, preferably the evenly distributed time intervals have lengths of 0.5 to 5 seconds.

8. The method according to claim 1 further comprising selecting the physical activity parameter corresponding to the physical activity.

9. A system for adjusting an overall duration of a physical activity of a biological object, the overall duration being pre-defined, the physical activity being evaluated based on a physical activity parameter, the comprising:
a. an interface for:
i. receiving a target information corresponding to the physical activity parameter;
ii. receiving the overall duration, the overall duration being a sum of a plurality of distributed time intervals;
b. at least one sensor for detecting a physical activity parameter information of the biological object, the physical activity parameter information being detected for each distributed time interval; and
c. a processing unit for:
i modifying the corresponding distributed time interval based on the detected physical activity parameter information and the target information, the corresponding distributed time interval being modified if the detected physical activity parameter information deviates from the target information; and
ii determining a new overall duration of the physical activity based on the modified distributed time interval and the received overall duration.

10. The system according to claim 9, wherein the processing unit is configured to modify the distributed time interval in proportion to the difference between the target information and the received physical activity parameter information.

11. The system according to claim 9, wherein the interface is further configured to receive a zone corresponding to the physical activity parameter, a lower and an upper boundary of the zone are defined by a minimum physical activity parameter threshold and a maximum physical activity parameter threshold respectively, wherein the processing unit modifies the distributed time interval if the received physical activity parameter information is in the zone.

12. The system according to claim 9, wherein the processing unit is further configured to determine a zone corresponding to the physical activity parameter, wherein a lower and an upper boundary of the zone are determined based on the target information and a pre-determined mathematical range corresponding to the target information, wherein the processing unit modifies the distributed time interval if the received physical activity parameter information is in the zone.

13. The system according to claim 9 further enables selection of the physical activity parameter corresponding to the physical activity.

14. A computer program product having computer readable program code embodied therein, when executed by a computer, causing adjusting an overall duration of a physical activity of a biological object, the overall duration being pre-defined, the physical activity being evaluated based on a physical activity parameter, in which the adjustment comprising:
a. receiving a target information corresponding to the physical activity parameter;
b. receiving the overall duration, the overall duration being a sum of a plurality of distributed time intervals;
c. receiving a physical activity parameter information of the biological object, the physical activity parameter information being received for each distributed time interval;
d. modifying the corresponding distributed time interval based on the received physical activity parameter information and the target information, the corresponding distributed time interval being modified if the received physical activity parameter information deviates from the target information; and
e. determining a new overall duration of the physical activity based on the modified distributed time interval and the received overall duration.

15. The computer program product according to claim 14 wherein the distributed time interval is modified in proportion to the difference between the target information and the received physical activity parameter information.
